(19) United States
(12) Patent Application Publication
(10) Pub. No.: US 2015/0241423 A1
(43) Pub. Date: Aug. 27, 2015

(54) IMMUNOASSAY USING ELECTROCHEMICAL DETECTION

(71) Applicant: MODE DIAGNOSTICS LIMITED, Glasgow (GB)

(72) Inventors: John Dilleen, Glasgow (GB); Louise Gray, Glasgow (GB); Paul Heaney, Glasgow (GB)

(21) Appl. No.: 14/436,291

(22) PCT Filed: Oct. 16, 2013
(86) PCT No.: PCT/EP2013/071590

§ 371 Date: Apr. 16, 2015

(30) Foreign Application Priority Data
Oct. 16, 2012 (GB) 1218555.9

(51) Int. Cl. G01N 33/543

(52) U.S. Cl. CPC G01N 33/54333; G01N 33/5438

(57) ABSTRACT

This invention relates to the detection of analyte in a sample using an electrochemical sensor that comprises a control sensing element, a detection sensing element and a magnet which selectively attracts magnetic beads to the detection sensing element relative to the control sensing element. The sensing elements produce a signal which is indicative of the amount of enzymatic label at the sensing element. In the presence of analyte in the sample exposed to the sensing elements, an immunocomplex is formed which comprises the analyte, the enzymatic label and a magnetic bead. The immunocomplex is attracted to the detection sensing element by the magnet. An increase in the amount of enzymatic label at the detection sensing element relative to the control sensing element is indicative of the presence or amount of analyte in the sample. Methods, sensors, devices and kits are provided.

### TECHNICAL FIELD

This invention relates to immunoassays for the detection of analyte in samples, and in particular, immunoassays which involve an electrochemical sensor.

### BACKGROUND

Sandwich assay formats are well-known for the immunological detection of analyte in a sample. In a sandwich assay, the analyte is sandwiched between a capture antibody immobilised on a solid phase and a labelled detection antibody which is free in solution. Sandwich assays generally require that excess labelled detection antibody is washed away before the detection step, so that the signal which is detected is produced by the antibody-analyte complex rather than unbound reagents.

Reducing the number of steps, including wash steps, required to perform an immunoassay would be desirable for many applications.

### SUMMARY OF THE INVENTION

This invention relates to an immunoassay which uses an electrochemical sensor to detect analyte in a sample in the presence of unbound reagents.

A first aspect of the invention provides a method for detecting analyte in a sample comprising:
a) providing an electrochemical sensor comprising;
   a control sensing element,
   a detection sensing element and
   a magnet which selectively attracts magnetic beads to the detection sensing element relative to the control sensing element,
   wherein upon exposure to a detection solution, each sensing element produces a signal which is indicative of the amount of an enzymatic label present in the detection solution at the sensing element,
b) exposing the electrochemical sensor to a detection solution,
   wherein the detection solution comprises;
   i) a sample being tested for the presence of analyte
   ii) a detection antibody reactive with said analyte which is attached to the enzymatic label, and
   iii) a separation antibody reactive with said analyte which is attached to a magnetic bead,
   such that, in the presence of analyte in the sample, a immunocomplex is formed in the detection solution which comprises the analyte, the detection and separation antibodies, the enzymatic label and the magnetic bead, and;
c) measuring the signals produced by the detection and control sensing elements, and
d) determining from said signals the amount of enzymatic label in the detection solution at the detection sensing element and the control sensing element,
wherein an increase in the amount of amount of enzymatic label at the detection sensing element relative to the control sensing element is indicative of the presence or amount of analyte in the sample.

A second aspect of the invention provides a method for detecting analyte in a sample comprising;
(i) providing an electrochemical sensor comprising control and detection sensing elements and a magnet which selectively attracts magnetic beads to the detection sensing element relative to the control sensing element,
each sensing element comprising a working electrode, a counter electrode, and optionally a reference electrode,
each working electrode having an electrically conductive matrix holding a first reagent and/or a second reagent, the second reagent being an oxidising agent or a precursor thereof for the first reagent;
   wherein the electrically conductive matrix is an electrically conductive carbon- or graphite-containing matrix or an electrically conductive porous matrix and a reaction between the first reagent and the oxidising agent is catalysable by an enzymatic label to provide a detectable signal at the working electrode;
(ii) exposing the control and detection sensing elements to a detection solution comprising;
   a) a sample for testing for the presence of analyte
   b) a detection antibody reactive with said analyte which is attached to an enzymatic label, and
   c) a separation antibody reactive with said analyte which is attached to a magnetic bead,
   such that, in the presence of analyte in the sample, a immunocomplex is formed in the detection solution which comprises the analyte, the detection and separation antibodies, the enzymatic label, and the magnetic bead, and;
(iii) maintaining a potential between the working electrodes and the counter electrode and/or the reference electrode, where present, in the detection and control sensing elements; and
(iv) measuring the currents passing between the test and control working electrodes and the counter and/or reference electrode where present, in the detection and control sensing elements,
an increase in the amount of current passing between the working electrode and the counter and/or reference electrode where present in the detection sensing elements relative to the amount of current passing between the control working electrode and the counter and/or reference electrode where present, in the control sensing element being indicative of the presence or amount of analyte in the sample.

A third aspect of the invention provides an electrochemical sensor for detecting analyte in a solution comprising;
a control sensing element,
a detection sensing element and
a magnet which selectively attracts magnetic beads in the solution to the detection sensing element relative to the control sensing element,
such that upon exposure to a solution, each said sensing element produces a signal which is indicative of the amount of enzymatic label present in the solution at the sensing element.

A fourth aspect of the invention provides the use of an electrochemical sensor, as described above, in method for detecting analyte.

A fifth aspect of the invention provides a sensing device comprising;
an electrochemical sensor according to the third aspect,
a sampler for accommodating a sample from an individual and, optionally, introducing said sample to the electrochemical sensor,
an electronic reader for determining from signals produced by the sensing elements of the electrochemical sensor the amount of analyte in the sample and providing an output indicative of said amount.

A sixth aspect of the invention provides kit for detecting an analyte comprising;
an electrochemical sensor according to the third aspect or a sensing device according to the fourth aspect,
a detection antibody reactive with said analyte which is attached or attachable to an enzymatic label;
a separation antibody reactive with said analyte which is attached or attachable to a magnetic bead, and;
optionally one or more buffers or other reagents.

### BRIEF SUMMARY OF THE DRAWINGS

FIG. 1 is a schematic diagram of detection in accordance with some embodiments of the invention.

FIG. 2 is a schematic of a sensing element as described herein.

FIG. 3 shows the detection of horseradish peroxidase (HRP) labelled magnetic beads in buffer. WE1 is the sensing element with the magnet and Electrode 2 is the sensing element without the magnet (as illustrated in FIG. 1).

FIG. 4 shows the detection of HRP labelled magnetic beads in buffer with detection antibody. WE1 is the sensing element with the magnet and Electrode 2 is the sensing element without the magnet (as illustrated on FIG. 1).

FIG. 5 shows the results of a C-reactive protein (CRP) bead assay with colorimetric detection

FIG. 6 shows the current vs. time transient for 0.0 μg/mL CRP bead assay with wash and electrochemical detection. WE1 is the sensing element with the magnet and WE2 is the sensing element without the magnet (as illustrated in FIG. 1).

FIG. 7 shows the current vs. time transient for 0.23 μg/mL CRP bead assay with wash and electrochemical detection. WE1 is the sensing element with the magnet and WE2 is the sensing element without the magnet (as illustrated in FIG. 1).

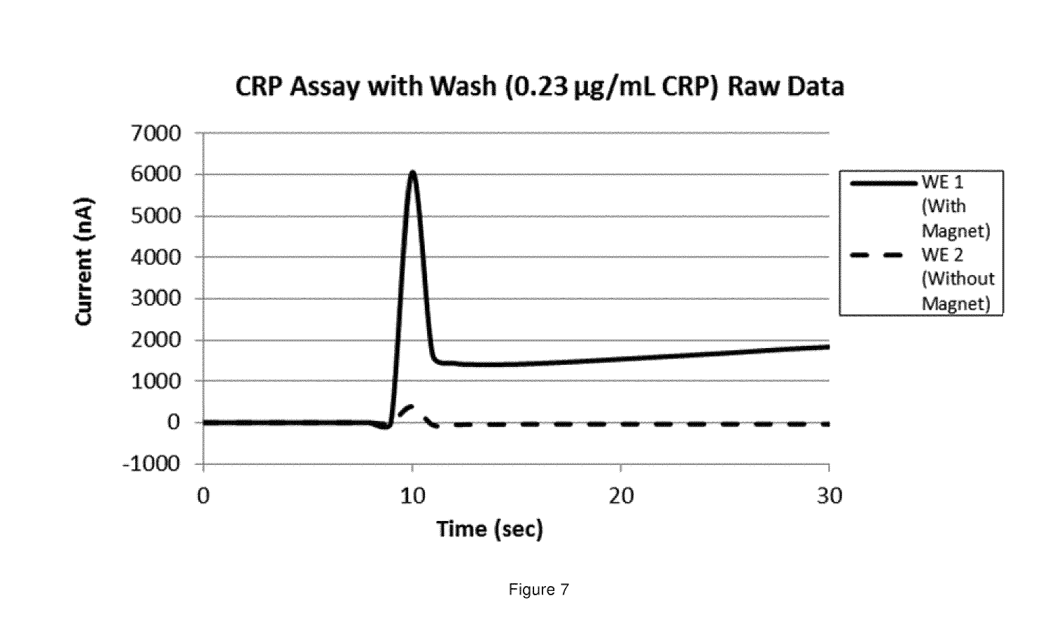

FIG. 8 shows CRP bead assay with wash and electrochemical detection. WE1 is the sensing element with the magnet and WE2 is the sensing element without the magnet (as illustrated in FIG. 1).

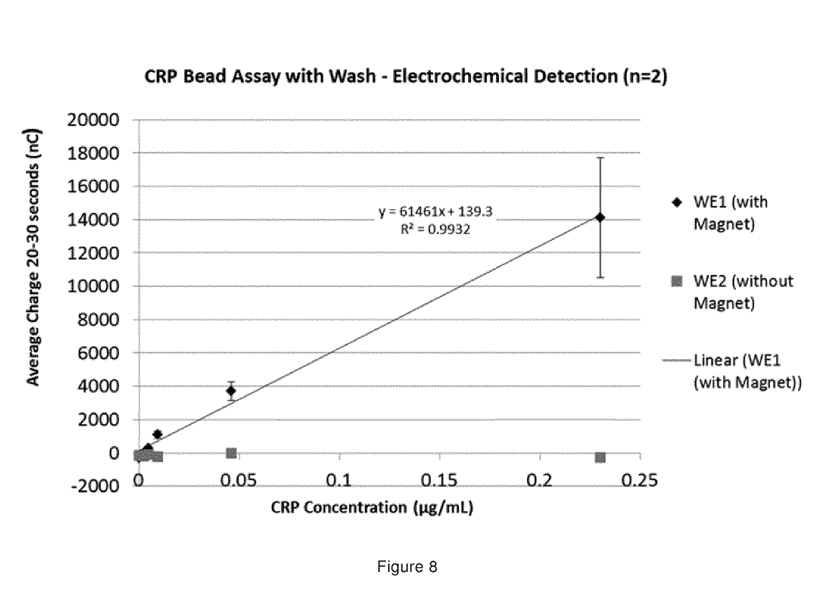

FIG. 9 shows the current vs. time transient for 0.0 μg/mL CRP bead assay (without wash) with electrochemical detection. WE1 is the sensing element with the magnet and WE2 is the sensing element without the magnet (as illustrated in FIG. 1).

FIG. 10 shows the current vs. time transient for 0.23 μg/mL CRP bead assay (without wash) with electrochemical detection. WE1 is the sensing element with the magnet and WE2 is the sensing element without the magnet (as illustrated in FIG. 1).

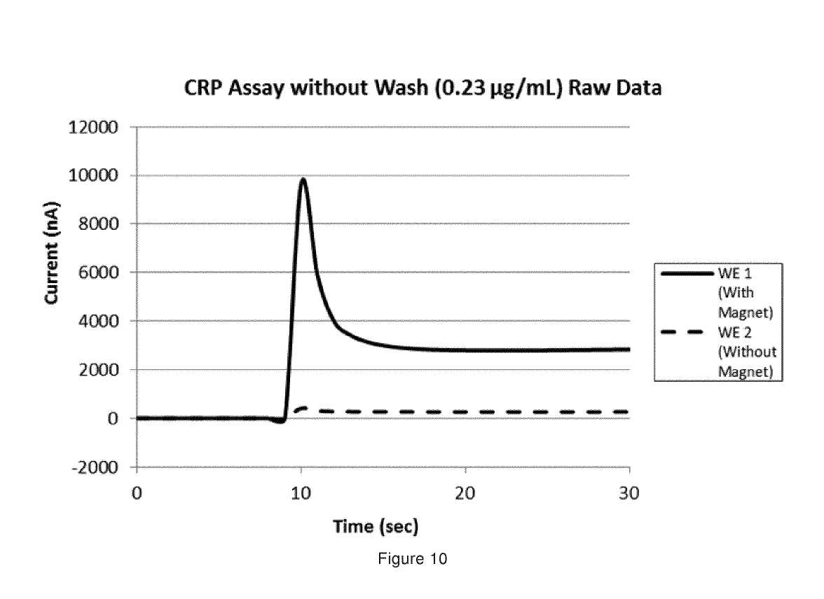

FIG. 11 shows CRP bead assay without wash with electrochemical detection. WE1 is the sensing element with the magnet and WE2 is the sensing element without the magnet (as illustrated in FIG. 1).

### DETAILED DESCRIPTION OF THE INVENTION

This invention relates to the use of an electrochemical sensor in an immunoassay to detect analyte in a sample.

The electrochemical sensor may be used to qualitatively or semi-quantitatively determine the amount of an analyte which is present in the environment to which the sensor is deployed. For example, the electrochemical sensor may be exposed to a detection solution which comprises a biological sample or a fraction thereof to detect the presence and/or measure the amount of analyte in the sample or fraction.

In some embodiments, the electrochemical sensor may be used to determine whether the level of analyte in a sample is at physiological level, i.e. the level expected in a healthy subject, or a clinical level, that is the level that is abnormal, or a level that is associated with a disease state.

The electrochemical sensor comprises a control sensing element and a detection sensing element. Suitable sensing elements for use in a sensor are described in WO2010/055306, the contents of which are incorporated by reference herein in their entirety.

Upon exposure to a detection solution, each sensing element produces a signal which is indicative of the amount of an enzymatic label which is present in the detection solution at the sensing element. For example, the signal may be indicative of the amount of an enzymatic label which is present in the detection solution in the electrolyte space which is defined by the electrodes of the sensing element.

The detection sensing element is associated with a magnet which selectively attracts magnetic beads in the detection solution to the detection sensing element relative to the control sensing element. In other words, magnetic beads are attracted more strongly by the magnet to the detection sensing element than to the control sensing element. Preferably, the magnet attracts magnetic beads in the detection solution to the detection sensing element and does not attract magnetic beads to the control sensing element.

The strength of the magnet may be adjusted according to the geometry and spacing of the electrodes, the thickness of the electrode sensor substrate, and the size of the magnetic beads in order to optimise the selective attraction of the magnetic beads to the detection sensing element. In some embodiments, suitable magnets may have a strength of 150 to 300 g, for example 160, 210, 250 or 290 g.

Suitable magnets for use in electrochemical sensors as described herein are readily available from commercial sources (e.g. Supermagnete DE) and include nickel plated (Ni—Cu—Ni) neodymium (NdFeB) magnets.

If analyte is present in the sample, then this analyte will be present in the detection solution. In the detection solution, the detection and separation antibodies bind to the analyte. This binding causes the formation of an immunocomplex which comprises the enzymatic label, the magnetic bead and the analyte. Because it comprises a magnetic bead, the magnet attracts this immunocomplex to the detection sensing element but not to the control sensing element. The immunocomplex is thus selectively attracted to the detection electrode and the enzymatic label which is present in the immunocomplex contributes to the signal which is produced by the detection sensing element but not the signal which is produced by the control sensing element. The presence of analyte in the detection solution therefore causes the signal from the detection sensing element to increase relative to the signal from the control sensing element.

The magnet is positioned relative to the sensing elements in order to selectively attract magnetic beads to the detection sensing element relative to the control sensing element. In other words, the position of the magnet generates a magnetic field in the sensor which is stronger at the detection sensing element than the control sensing element, such that magnetic beads are more strongly attracted to the detection sensing element than the control sensing element. Preferably, the magnet is positioned to generate a magnetic field in the sensor at the detection sensing element but not at the control sensing element, such that magnetic beads are attracted to the detection sensing element but not to the control sensing element.

For example, the magnet may be positioned at the detection sensing element, e.g. in, on or under the detection sensing element, such that magnetic beads attracted to the magnet arrive at the detection sensing element; or the magnet may be positioned near, adjacent or in proximity to the detection sensing element, e.g. within 5 mm of the sensing element, to achieve the same effect.

The control sensing element and detection sensing element are separated in the sensor, such that neither sensing element is able to detect enzymatic label which is at the other sensing element i.e. the signal from the detection sensing element is independent of the signal from the control sensing element.

The sensing elements may be separated sufficiently to prevent diffusion of electro-generated chemical species between the sensing elements over the duration of the analysis. A suitable separation of sensing elements for any particular analysis may be readily determined by a skilled person. In some embodiments, the sensing elements may be separated by at least 1 mm for every minute of the analysis. Typically, the sensing elements may be separated by at least 1 mm, 2 mm, 3 mm, 4 mm, or 5 mm.

The electrochemical sensor may further comprise a detection chamber which accommodates the detection solution. The sensing elements may be positioned in the detection chamber, such that they are exposed to detection solution accommodated in the detection chamber.

In some embodiments, the sensing elements may be located in separate detection chambers. For example, an electrochemical sensor may comprise first and second detection chambers. The first and second detection chambers may accommodate portions of the detection solution. The detection sensing element may be positioned in the first detection chamber, such that it is exposed to detection solution in the first detection chamber and the control sensing element may be positioned in the second detection chamber, such that it is exposed to detection solution in the second detection chamber.

The control sensing element and/or detection sensing element may be fixed or fixable to a solid support. The solid support may retain the sensing elements in position, for example in the reaction chamber and maintain their separation. In some embodiments, the solid support may define or partly define the interior surface of the detection chamber.

Each sensing element may comprise a working electrode, a counter electrode and optionally a reference electrode. In some embodiments, a combined reference/counter may be used e.g. for qualitative measurement, semi-quantitative measurements or quantitative measurements in predictable samples. The electrodes are connectable to a power source. Suitable electrodes are described in WO2010/055306, the contents of which are incorporate wherein in their entirety.

The working electrode, counter electrode and optionally the reference electrode, define an electrolyte space. In use, the electrodes are in electrical contact with the detection solution in the electrolyte space and the amount of enzymatic label which is present in the electrolyte space defined by the electrodes of the sensing element is determined.

The working electrode may comprise one or more reagents which are catalysable by the enzymatic label attached to the detection antibody to provide a detectable signal at the working electrode. The reagents may be held at the working electrode in an electrically conductive matrix. For example, a working electrode may have an electrically conductive matrix holding a first reagent and/or a second reagent, the second reagent being an oxidising agent or a precursor thereof for the first reagent; wherein a reaction between the first reagent and the oxidising agent is catalysable by the enzymatic label to provide a detectable signal at the working electrode.

Suitable electrically conductive matrices include an electrically conductive carbon- or graphite-containing matrix or an electrically conductive porous matrix, for example a carbon paste.

The choice of first and second reagents may depend on the enzymatic label which is being employed. The first reagent may be reactable with the second reagent in the presence of the enzymatic label.

Suitable first reagents may be, or may comprise, a compound selected from tetramethylbenzidine, alpha guaiaconic acid, 2,2′-azino-bis(3-ethylbenzothiazolidine-6-sulphonic acid), hydroquinone, phenylenediamine, o-dianisidine, o-tolidine (dimethylbenzidine), 6-methoxyquinoline, and 3,3′-diaminobenzidine, 3-amino-9-ethylcarbazole, preferably tetramethylbenzidine. Preferably the first reagent is held in the electrically conductive matrix. For example, the first reagent may be present in the electrically conductive matrix at 1 to 15 wt %, preferably 2-9 wt % or about 5 wt %.

The second reagent may be an oxidising agent or a precursor thereof. It may be reactable with the first reagent in the presence of the enzymatic label. The second reagent may be held in the electrically conductive matrix.

Suitable second reagents for the detection of a peroxidase enzymatic label may comprise hydrogen peroxide or a precursor thereof. For example, the second reagent may be, or may comprise, urea peroxide or sodium perborate, preferably sodium perborate. Preferably, the second agent is hydrogen peroxide. Therefore, the first reagent is preferably a compound that reacts with hydrogen peroxide in the presence of the peroxidase enzymatic label.

Suitable second reagents for the detection of a glucose oxidase enzymatic label may comprise glucose or a precursor thereof.

In some preferred embodiments, working electrodes in sensing elements for the detection of a peroxidase enzymatic label may comprise a carbon paste (CP) matrix which holds tetramethylbenzidine (TMB) and perborate (PER).

In some embodiments, the electrically conductive matrix of the working electrode may hold a single reagent (i.e. a first reagent only). A reaction between the reagent and the enzymatic label provides a detectable signal at the working electrode without the need for a second reagent. This may be useful, for example, in the detection of an alkaline phosphatase enzymatic label. Suitable reagents for the detection of alkaline phosphatase include 1-naphthyl-phosphate; 5-bromo-4-chloro-3-indolyl phosphate (BCIP); hydroquinone diphosphate; phenolphthalein phosphate; 4-aminophenyl phosphate; 3-idoxyl phosphate and phenyl phosphate.

Other preferred formats for working electrodes in sensing elements are described in WO2010/055306.

In addition to the first reagent and/or second reagent, the working electrodes in the sensing elements may optionally comprise one or more further additives. For example, the working electrode may further comprise a wetting additive, which may optionally be held in the electrically conductive matrix. Suitable wetting additives may include polyvinylpyrrolidone, Triton X, and/or tween. The wetting additive may present in the electrically conductive matrix at 0.005-0.25 wt %.

Other suitable working electrode additives are described in WO2010/055306.

In some embodiments, the working electrodes in the sensing elements may be wholly or partially coated over at least part of their surface. The coating on the electrode is preferably soluble in the detection solution and is removed from the working electrode by dissolution upon exposure to the detection solution. Suitable coatings include water-soluble polymers, such as polyalkylene glycol, for example polyethylene glycol; cellulosic polymers, for example hydroxyalkylcellulose including hydroxyethylcellulose and hydroxypropyl methylcellulose; sucrose or other polysaccharides, for example chitosan; and vinyl polymers, for example poly(vinylpyrrolidone) and poly(vinylpyrrolidone)-(vinyl acetate) copolymer.

The electrochemical sensor comprises a counter electrode. The counter electrode may be of sufficient size to carry the currents from the working electrodes and may typically have an effective electroactive area of at least 1× the combined area of the other electrodes in the sensor elements, thereby ensuring that the current flow from both of the working electrodes is not limited. The counter electrode is connectable to a power source. Preferably, the counter electrode is connected to the power source when the counter electrode is used as a reference electrode.

There are no specific limitations on the type of counter electrode that may be used in the electrochemical sensor of the invention and suitable counter electrodes for use in sensing elements as described herein are well known in the art. Preferred electrode materials include carbon, steel and platinum. Steel and carbon are the most preferred electrode material for use in disposable and one shot sensors and apparatus owing to their relatively low cost. For example, a suitable counter electrode may be carbon.

In some embodiments, a reference electrode may be included in the electrode device of the invention. The reference electrode may be a standard silver/silver chloride electrode. The reference electrode may be a pseudo reference electrode, which is operable as a reference electrode in the presence of a suitable buffer comprising appropriate ions. In one embodiment, the pseudo reference electrode may be a silver-based electrode that is obtained, or is obtainable from, a silver electrode that is treated with about 1% aqueous FeCl₃ solution. The electrode may be washed before and/or after the treatment. A pseudo reference may, for example, also be screen printed. The screen printing of Ag/AgCl reference electrodes is well-established in the art (e.g. for use in glucose biosensors).

In some embodiments, a combined counter and reference electrode may be employed.

Three electrode formats may be useful, for example, in providing greater accuracy and precision for low end detection, whilst two electrode formats may be preferred for high end and qualitative analysis.

Electrodes for use in sensing elements as described herein may be produced using standard techniques. For example, the electrodes may be screen-printed onto carbon contact on an insulating solid e.g. a polyester solid, or may be screen printed directly onto the insulating solid.

Both sensing elements may be adapted for electrical connection to a voltage supply, such as a potentiostat. In some embodiments, the sensing elements in the sensor may be electrically connected to a connector, such as a port, plug or socket, which is connectable to a voltage supply, in an electronic reader, as described below.

The electrochemical sensor determines the presence or amount of analyte present in a detection solution to which it is exposed.

The detection solution may comprise;
a sample being tested for the presence of analyte,
a detection antibody reactive with the analyte which is attached to an enzymatic label;
a separation antibody reactive with the analyte which is attached to a magnetic bead, and;
optionally one or more buffers or other reagents.

The analyte may be any molecule, complex, aggregate or cell whose presence or amount in a sample requires detection or measurement. Suitable analytes include two or more antigenic epitopes which allow the analyte to be bound by two antibodies simultaneously (i.e. a detection antibody and a separation antibody as described herein).

The analyte may be a protein, nucleic acid, carbohydrate, or lipid or combinations thereof, cells or organic molecules, such as bacteria, viruses and natural or synthetic chemical molecules.

Suitable analytes include C reactive protein, haemoglobin, calprotectin, Chlamydia, Lactoferrin, Elastase, E. coli, H. pylori, Prostate Specific Antigen, β-catenin, Human Chorionic Gonadotropin, Insulin-like growth factor 1 (IGF-1) and Anti-Müllerian hormone.

The biological sample which is tested for analyte may be any biological fluid in which analyte is to be detected or measured. For example, the biological fluid may be blood, serum, plasma, stool, urine, lumen, digestive enzymes, wound fluid, semen, intestinal fluid, lymph, saliva, sweat, cerebrospinal fluid, or tears.

The biological sample may be processed, fractionated, purified and/or partially purified before exposure to the electrochemical sensor. For example, red blood cells may be removed from a whole blood sample using a plasma separation membrane if red blood cells or haemoglobin are not being detected or quantified.

The detection antibody is an antibody which specifically binds to the analyte.

Suitable antibodies for any analyte of interest are readily available in the art and may be produced by routine techniques or obtained from commercial suppliers.

The detection antibody may be linked or linkable to an enzymatic label.

The enzymatic label catalyses the oxidation of the reagents held at the working electrode to produce a detectable signal. For example, the working electrode may hold first and optionally second reagent in an electrode electrically conductive matrix as described above and the enzymatic label may catalyse the oxidation of the first reagent, optionally by the second reagent. The oxidised form of the first reagent may then be reduced at the electrode to provide the detectable signal at the electrode. Suitable reagents for use in the working electrodes to detect each enzymatic label are described in more detail above.

Suitable enzymatic labels are well known in the art and include peroxidase, glucose oxidase and alkaline phosphatase. Preferably, the label is a peroxidase, such as horseradish peroxidase (HRP).

Enzymatic labels may be produced using standard recombinant techniques or obtained from a commercial supplier (for example Acris Antibodies; Santa Cruz; Abcam Ltd, UK; R&D Systems; DAKO; Invitrogen, USA).

The detection antibody may be attached directly to the enzymatic label or indirectly through a linker molecule. A linker molecule may be covalently bound to the detection antibody and enzymatic label or may be non-covalently bound to one or both the detection antibody and enzymatic label. For example, the enzymatic label maybe conjugated to a secondary antibody which binds to the detection antibody. Binding of the secondary antibody attaches the enzymatic label to the detection antibody.

Suitable methods for attaching or conjugating a detection antibody or a secondary antibody to an enzymatic label are well known in the art.

The separation antibody is an antibody which specifically binds to the analyte. The separation antibody binds to a different antigenic epitope to the detection antibody and does not compete with the detection antibody for binding to the analyte. In other words, the detection and separation antibodies may bind to the analyte at the same time to form a complex comprising the analyte, the detection antibody and the separation antibody.

As described above, antibodies for any analyte of interest are readily available in the art and may be produced by routine techniques or obtained from commercial suppliers (Acris Antibodies; Santa Cruz; Abcam Ltd, UK; R&D Systems; DAKO; Invitrogen, USA).

The separation antibody is linked to a magnetic bead.

Magnetic beads are ferromagnetic particles which are readily conjugated to biomolecules. Suitable beads may have a diameter of about 0.1 to 10 μm, preferably 1 μm. The use of magnetic beads is well known in the art and suitable beads are available from commercial suppliers (e.g. Life Technologies, USA; Chemcell GmbH, DE). Suitable beads may, for example, comprise a non-porous silica matrix surrounding a maghemite core.

The separation antibody may be attached directly to the magnetic bead or indirectly through a linker molecule. A linker molecule may be covalently bound to the separation antibody and magnetic bead or may be non-covalently bound to one or both the separation antibody and magnetic bead. For example, the magnetic bead maybe conjugated to a secondary antibody which binds to the separation antibody. Binding of the secondary antibody attaches the magnetic bead to the separation antibody

Suitable methods for linking an antibody to a magnetic bead are well known in the art.

The detection and separation antibodies are selected specifically for the target analyte. The antibodies are paired to ensure that different epitopes on the analyte are targeted, so that both the antibodies can bind to create an immunocomplex comprising the antibodies and the analyte.

Preferably, the detection and separation antibodies are monoclonal but, appropriately matched polyclonal antibodies may be useful in some applications. Preferably, the antibodies are isolated and/or purified (e.g. to >95%) to facilitate labelling.

Other immunoassay reagents include cell lysing agents, such as saponin. Other standard immunoassay reagents may be used as necessary.

The electrochemical sensor detects the presence or amount of immunocomplexes comprising analyte, label and magnetic beads in the detection solution.

The detection solution may be produced by any suitable technique or process.

In some embodiments, the sample and immunoassay reagents may be admixed in an initial assay solution, such that immunocomplexes comprising the analyte, label and magnetic beads form in the assay solution, if the analyte is present in the sample. The assay solution may then be treated, for example by lowering the pH, to produce the detection solution.

The sample may be admixed with the detection and separate antibodies and other immunoassay reagents to produce the assay solution, with mixing as required. The immunoassay reagents may be admixed simultaneously or sequentially with the sample. For example, the sample may be admixed with the detection antibody followed by the separation antibody or vice versa

The assay solution may be incubated under conditions which facilitate the binding of antibodies to analyte in the sample.

The assay solution may be incubated at a temperature of around 37° C. Alternatively the sample may be at room temperature. For example, the assay solution may be incubated at a temperature in the range 5 to 45° C., 10 to 30° C., or preferably 18 to 25° C. The temperature of the assay solution may be adjusted to bring it to the preferred temperature. For example, assay solution may be cooled or allowed to cool from physiological temperature to room temperature.

In some embodiments, the assay solution may be incubated for 1-10 minutes at pH 7.4 and ambient temperature. Typical laboratory assay incubation times include about 2 hours incubation; however this may be reduced to 1-10 minutes, for example for rapid home testing

Following the formation of complexes comprising the label and the magnetic bead in the presence of analyte, the assay solution may further treated to produce the detection solution. For example, the pH of the assay solution may be reduced to a pH of 3 to 7, preferably 4 to 5, preferably about 5. This may be achieved by adding a suitable buffer to the assay solution. Alternatively, this may be achieved in a cartridge format by drying down reagents in specific zones to control the pH of the buffer e.g. citric acid.

The presence of enzymatic label in the detection solution at a sensing element causes the sensing element to produce a signal. The enzymatic label which elicits the signal may be part of an immunocomplex comprising the analyte or may be present as a part of an unbound detection antibody conjugate. The amount of enzymatic label which is present in the detection solution at the detection sensing element and the control sensing element is then determined from the signals produced by the sensing elements.

The presence of analyte in the sample leads to the presence of immunocomplexes in the detection solution which comprise analyte, magnetic bead and enzymatic label. These immunocomplexes are attracted to the detection sensing element by the magnet but are not attracted to the control sensing element. This causes the amount of enzymatic label in the detection solution at the detection sensing element to increase relative to the amount at the control electrode. The relative amounts of enzymatic label in the detection solution at the detection sensing element and the control sensing element are therefore indicative of the presence or amount of analyte in the sample.

An increased amount of enzymatic label at the detection sensing element relative to the control sensing element may be indicative of the presence of analyte in the sample. The difference in the amount of enzymatic label at the detection sensing element relative to the control sensing element may be indicative of the amount of analyte in the sample.

The sensing elements may produce an electrical signal which is indicative of the amount of enzymatic label in the detection solution at the sensing element. The signal may be an amperometric or a potentiometric signal. For example, the signal may be the potential difference between the working electrode and the counter and/or reference electrode where present, at a constant or zero current, or more preferably, the signal may be the current passing between the working electrode and the counter and/or reference electrode where present, at a constant potential.

In some preferred embodiments, the amount of enzymatic label at a sensing element may be determined by;
(iii) maintaining a potential across the working electrodes and the counter electrodes and/or the reference electrodes, where present; and
(iv) measuring the currents passing between the test and control working electrodes and the counter and/or reference electrodes where present.

The amount of current passing between the working electrode and the counter and/or reference electrode where present is indicative of the amount of enzymatic label in the assay solution at the sensing element.

An increased amount of current passing between the working electrode and the counter and/or reference electrode where present, at the detection sensing element relative to the amount of current passing between the working electrode and the counter and/or reference electrode where present, at the control sensing element is indicative of the presence of analyte in the sample. The difference in the amount of current at the detection sensing element relative to the control sensing element may be indicative of the amount of analyte in the sample.

The electrochemical sensor may be contained in a housing or cartridge.

The housing or cartridge may be disposable.

The housing or cartridge may contain one or more reagent reservoirs, assay chambers and liquid transfer systems or conduits to facilitate the mixing of the sample with the immunoassay reagents, the production of the detection solution and the transport of the sample to the detection chamber for exposure to the electrochemical sensor.

Preferably, the reagent reservoirs, assay chambers and liquid transfer systems or conduits are arranged such fluid is transferred to the electrochemical sensor by gravitational flow.

In some embodiments, the sensing elements of the sensor may be retained within a cavity in the cartridge. Coating material may be packed into the cavity, thereby at least partially covering the sensing elements within the cavity. Upon hydration by the detection solution, the coating material may dissolve to expose the electrodes of the sensing elements to the detection solution.

The cartridge may comprise one or more fluid transfer conduits to convey the sample or a fraction or portion thereof from the sampler to the detection chamber of the sensor. In some embodiments, the cartridge may further comprise an assay chamber in which the sample is admixed and incubated with the immunoassay reagents before detection in the detection chamber. For example, sample may be transported from the sampler by a first fluid transfer conduit to an assay chamber where it is admixed with immunoassay reagents to form the assay solution. The assay solution may be incubated in the assay chamber and transported by a second fluid transfer conduit to the detection chamber for exposure to the sensor. The assay solution may be treated to produce the detection solution in the assay chamber, fluid transfer conduit or the detection chamber.

The cartridge may comprise one or more immunoassay reagents. For example, the cartridge may comprise the detection antibody, the separation antibody and one or more buffers or other immunoassay regents.

Immunoassay regents in the cartridge may include an assay buffer for mixing the immunoassay reagents and/or sample to produce an assay solution.

Immunoassay regents in the cartridge may also include a detection buffer for mixing with the assay solution to produce a detection solution. The detection buffer may, for example, reduce pH so that the detection solution has a lower pH than the assay solution and is compatible with the detection of the enzymatic label by the electrochemical sensor. Preferably, the detection buffer comprises a sufficient concentration of chloride ions for the pseudo-reference electrode in the sensing element to approximate the behaviour of a true reference electrode.

The immunoassay reagents in the cartridge may be stored in reservoirs within the cartridge prior to use or may be stored in lyophilised form and may be solubilised to produce the assay or detection solution following the introduction of the sample to the cartridge.

A reagent reservoir in the cartridge may release a reagent upon contact with the sample or the detection or assay solution. The reservoir material may, at least in part, be soluble and is preferably soluble upon hydration, thereby to release the reagent into the sample. The reservoir is preferably composed of a water-soluble polymer. Suitable water-soluble polymers are those polymers described herein for use as a coating material for the electrode.

A reservoir may be located in close proximity to the sensing elements, and may for example be located adjacent the electrolyte space of the detection and/or control sensing elements. A reagent contained within the reservoir may therefore be made available to the assay solution or detection solution prior to and during electrochemical analysis. Alternatively, the reservoir may be located in close proximity to the assay and/or detection chamber(s).

The cartridge may further comprise a heating element and/or a mixer to facilitate interaction and mixing between the one or more immunoassay reagents and the sample in the assay chamber, detection chamber and/or fluid transfer conduits.

The cartridge may be adapted to connect to or engage with an electronic reader.

The cartridge may include a connector, such as a plug, socket or port, which provides an electrical connection to the electronic reader. The connector allows the electrical connection of the electrochemical sensor to the electronic reader, such that power can supplied to the sensor and signals from the sensor can be analysed, processed and/or recorded in the reader. The connector may be linked to the sensing elements by wiring or other circuitry contained in the cartridge.

An electrochemical sensor or cartridge comprising an electrochemical sensor may be part of a sensing device.

A sensing device may comprise;
an electrochemical sensor as described above,
a sampler suitable for storing and/or sampling a biological sample from a subject, and
an electronic reader for determining the amount of analyte in the sample from signals produced by the sensing elements of the electrochemical sensor and providing an output indicative of said amount.

The electrochemical sensor may be contained within a cartridge for use in a sensing device.

The sensing device may be adapted to analyse a sample from a subject. The sensing device may be a handheld device and may be adapted for use by a user who is not a clinician or a qualified technician. The sensing device may be provided for use by a private individual as part of a home test kit.

The sensing device is not limited in shape, size or construction. Preferably the sensing device is adapted for use with a biological sample, and is adapted for use in electrochemical analysis of that sample. In one embodiment the sensing device is in the form of a body suitable for holding by hand.

The sampler may be adapted to remove a sample from a biological fluid from an individual and/or accommodate a sample of biological fluid.

The form of the sampler depends on the sample and the test to be performed. For example, the sampler may comprise a wick for urine or other bodily fluid samples, a capillary fill chamber for blood or other bodily fluid samples, stool sampler, skin cell scraper, or a swab for samples from cervix, endocervix, urethra, mouth, tongue or nose.

The sampler may comprise an element which facilitates extraction of the sample of biological fluid from an individual. For example, the sampler may comprise a lancet which enables the individual to prick their skin (e.g. at the finger, ear etc), or a urine collection device that voids the first flow of urine e.g. excludes the first 10 ml.

In some embodiments, the sampler may comprise a sample chamber for the accommodation of a sample of biological fluid. Preferably, the sampler is disposable.

The sampler may be connectable with the cartridge, such that sample accommodated in the sampler is delivered to the cartridge.

The sampler may comprise one or more processing elements which allow the separation, purification and/or fractionation of the sample. For example, the sampler may comprise a plasma separation membrane for the analysis of whole blood.

In some embodiments, one or more of the immunoassay reagents may be contained in the sampler. For example, the detection antibody may be immobilised within the sampler in lyophilised form. The introduction of the sample to the sampler may solubilise the detection antibody.

The sampler may be integral with the sensing device and may be removable therefrom. Thus, there is provided a single piece of equipment for sampling and analysing a sample.

The electronic reader may comprise an electronic display, for example a liquid crystal display (LCD), which is capable of providing a visual indicator as to the result of the analysis. The indicator may be a word and/or a symbol. The electronic display provides greater certainty as to the result displayed, and the display is not susceptible to subjective interpretation. Such interpretation is a particular disadvantage of tests where a positive result is indicated as a colour change. The change may be difficult to visualise, and may not be uniform, thereby providing an inconclusive or uncertain result to the user. For example, an electronic display may provide a visual result, for example a numerical value, indicative of the amount of analyte in the sample.

The electronic reader may further comprise a voltage supply (or power supply) to control the sensing elements in the sensor. The voltage supply is preferably adapted to supply a constant bias between the working electrodes and the counter electrodes or the reference electrodes, where present, of the detection and control sensing elements. Preferably, the voltage supply is adapted to supply a constant bias in the range −1 to +1 volt, preferably −0.4 to +0.4 volts, more preferably +0.03 volts vs screen printed Ag/AgCl reference electrode between the working electrodes and the reference or counter electrodes.

The electronic reader may further comprise a processor to determine the amount of analyte in the sample from the signals produced by the electrochemical sensor. The electronic reader may further comprise a memory for recording and storing data from the electrochemical sensor and/or a counter to indicate the number of tests taken and/or the number of tests remaining.

The electronic reader may comprise a connector, for example a plug or socket, for connection to the connector on the cartridge.

The electronic reader may be further provided with an alarm that indicates to a user when a further test on a new sample should be performed. The alarm may be a visual or audible alarm or both.

Data may be downloadable from the electronic reader, for example through add-on hubs connectable to cartridge port of the reader or a separate port, such as a USB port, or through a wireless connection, such as Bluetooth or wi-fi.

The sensing device may be used to indicate the presence of analyte over a series of tests. Repeat experiments minimise the chance of false positive results. The sensing apparatus may be adapted for a series of repeat experiments. Thus the sensing apparatus may have an electrochemical sensor that is usable two or more times. Alternatively, the device may be provided with two or more electrochemical sensors, where each sensor is provided for one of the series of experiments.

The sensing device may be supplied as part of a kit, which may further comprise immunoassay reagents, diluents or buffers as described herein, or mixtures suitable for generating a diluent, for example by addition of water.

Immunoassay reagents, diluents and buffers may be provided in a kit as solids or gels for make-up into a liquid form, for example by addition of water.

A kit may further comprise other components for use in obtaining and analysing a sample using the electrochemical sensor, such as a lancet device, urine collection vessel, stool collection device (e.g. toilet sling or platform), add-on plug-in component to allow connectivity to the electronic reader (e.g. usb, bluetooth, wi-fi) and/oro hygienic disposal bag.

The kit may include a set of operating instructions. The instructions may relate to the use of the sensing device, the use of the storing and/or sampler, and the interpretation of the sensing device results. The operating instructions may be in paper form, on an electronic carrier or available or downloadable from a website, whose address is provided.

Other aspects and embodiments of the invention provide the aspects and embodiments described above with the term “comprising” replaced by the term “consisting of” and the aspects and embodiments described above with the term “comprising” replaced by the term “consisting essentially of”.

Modifications of these embodiments, further embodiments and modifications thereof will be apparent to the skilled person on reading this disclosure, and as such these are within the scope of the present invention.

It is to be understood that the application discloses all combinations of any of the above aspects and embodiments described above with each other, unless the context demands otherwise. Similarly, it is possible to combine preferred and/or optional features singly or together with any of the other aspects, unless the context demands otherwise.

Various further aspects and embodiments of the present invention will be apparent to those skilled in the art in view of the present disclosure.

All documents and database entries which are mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments of the invention which are described. Thus, the features set out above are disclosed for use in the invention in all combinations and permutations. Certain aspects and embodiments of the invention will now be illustrated by way of example and with reference to the figures and tables described herein.

### EXAMPLES

The following examples are provided solely for illustrative purposes and are not intended to limit the scope of the invention, as described herein.

1. Assay Concept

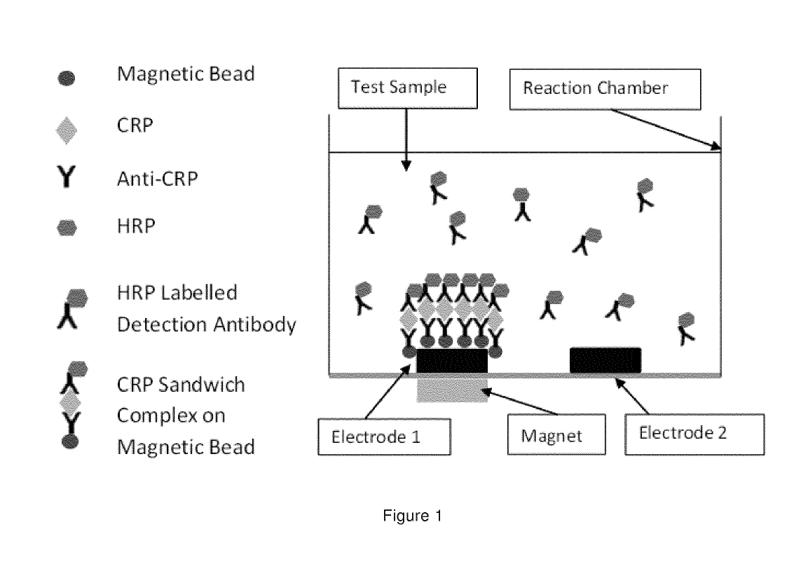

The electrochemical sensor described herein distinguishes between the actual signal due to the analyte and the background signal due unbound detection antibody still in solution. In examples, the peroxidase HRP has been selected as the enzymatic label for detection by the sensor. In order for the HRP to react with the electrode chemistry, it must be in very close proximity of the sensor. With no external forces the reaction would be diffusion dependent. This would give a slow response with low magnitude signals in direct relation to analyte concentrations. The assay is carried out on magnetic micro-beads to enable the antibody-analyte complex to be brought to the electrode using the force of a magnetic field. The system uses two identical functional electrodes: electrode 1 has a magnet behind it and electrode 2 has no magnet. The beads are transported to electrode 1 in the magnetic field. Electrode 2 is used to record the background signal cause due to unbound detection antibody. The difference between the two signals can be used to calculate the concentration of the analyte in solution. This concept is illustrated in FIG. 1.

In FIG. 1, the analyte (CRP) is sandwiched between a capture antibody on a magnetic bead and a detection antibody that is labelled with HRP. A magnetic field is used to bring the magnetic beads to a functionalised electrode that is sensitive to HRP. A second electrode is used to detect the background signal present in the test solution due to unbound HRP labelled detection antibody which free in solution. Both of the electrodes are identical except of the addition of a magnet behind electrode 1.

2. Methods

2.1 Electrochemical Sensor

For the following experiments, a sensing device was split into two parts: the reaction chamber and the sensing elements.

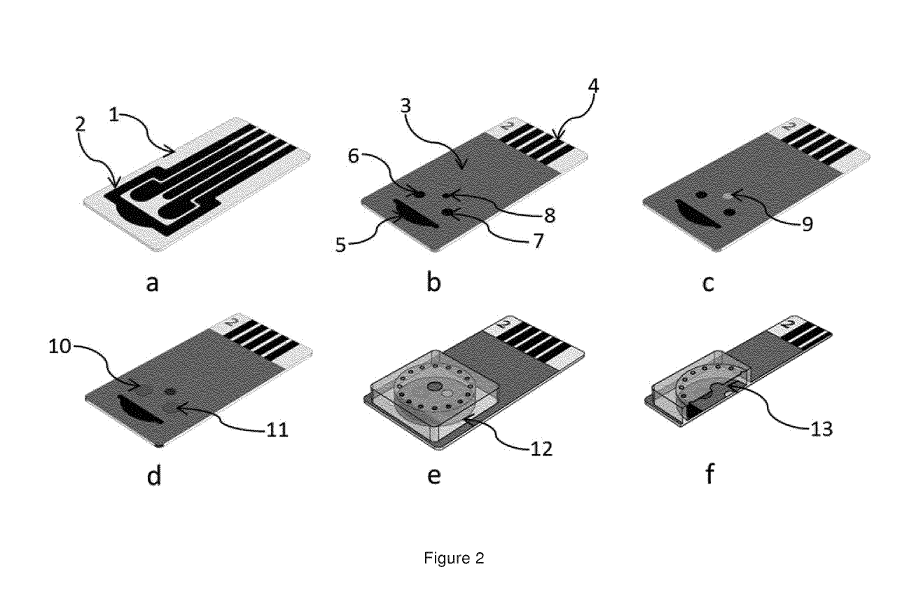

The assay was completed in a reaction chamber. A well of a 96 well plate was used as the reaction chamber for each test. The assay solution was then transferred into a well on the sensing electrode. The sensing electrode was powered and controlled by a potentiostat and data was collected using a data acquisition device. Each sensing element consisted of a standard electrode electrochemical cell which was either a two electrode (Working & Counter) or a three electrode (Working. Reference and Counter) format, depending on the predictability or complexity of the sample (e.g. stool is unpredictable and varies from sample to sample). The electrodes were screen printed on 350 micron Polyester Film (Kemafoil®, mtsl w, Coveme. UK) as shown in FIG. 2. The counter electrode was screen printed carbon. The reference electrode was screen printed Ag/AgCl on a screen printed carbon contact. The working electrodes were CP/TMB/PER blend laid on a screen printed carbon contact. The detection sensing element (Electrode 1 in FIG. 1) had a magnet behind it whereas the control sensing element (Electrode 2 in FIG. 1) had no magnet. A 14 mm diameter well was defined on the surface of the sensing element to hold the test solution for the duration of electrochemical testing with a 250 micron double sided adhesive polyester tape.

2.2 Buffers and Reagents

Assay Buffer:
PBS Buffer (Product Code: P5368, Sigma, UK)

Electrochemical Buffer:
1× Buffer A (pH 5.0) (Made in house)
The concentration of chloride ions in the buffer is sufficient for the pseudo-reference electrode to approximate the behaviour of a true reference electrode.
Buffering strength is sufficient to ensure that the s a pH value close to 5.0 (the pH of assay solution is 7.4).

Assay Dilution:

Assays were carried out in 107.7 μL Assay buffer then diluted with 300 μL Electrochemical Buffer to give a 407.7 μL test solution at pH 5.

3. Testing of Detection Concept

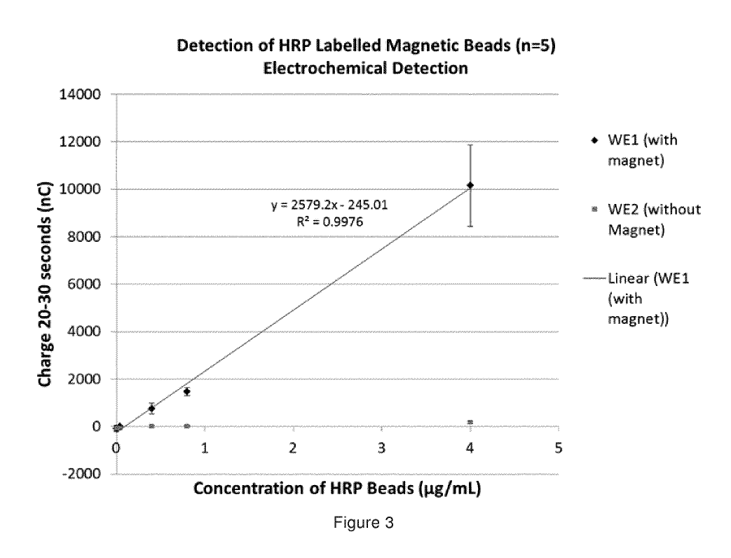

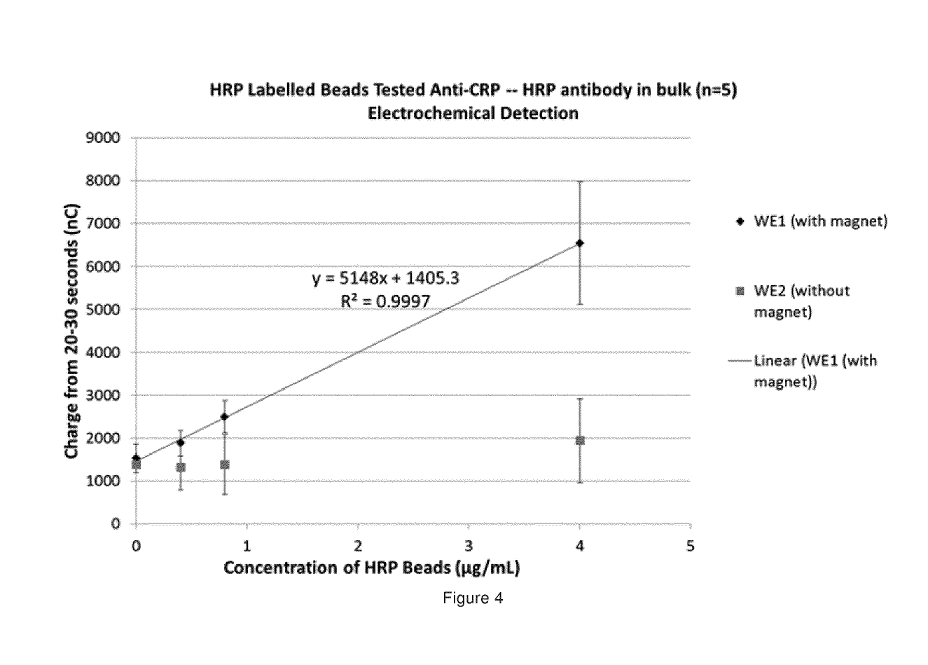

Magnetic micro-beads were labelled directly with HRP. A range of concentrations of these beads were tested with the electrochemical sensor. Firstly, the beads were tested in standard electrochemical buffer solution (FIG. 3). Secondly, the beads were tested in electrochemical buffer containing HRP labelled detection antibodies at the same concentration as used in the full assay to mimic the background signal that would be present when a full assay is being tested (FIG. 4). The data from these tests demonstrated that the detection concept works very well. In both tests, the signal derived from the HRP labelled beads gave a linear trend.

4. Bead Assay

A CRP assay was design and tested as a method to demonstrate the technique. The assay was designed with Magnetic Microbeads as the solid phase. Colourimetric detection was used to develop and verify the assay.

The analyte was incubated for 1 hour with the detection antibody which was labelled with HRP in a reaction chamber (a well of a 96 well plate). Magnetic beads that were conjugated with capture antibody were then added to the reaction chamber and the solution was incubated for an additional hour. The beads were washed 3 times then re-suspended in solution. The bead solution was then transferred from the reaction chamber to a clean detection chamber. The beads were separated from the test solution and a colourimetric solution was added and incubated for 10 minutes. A stop solution was added and the 96 well plate and the beads were removed (as the colour of the beads causes a background signal at 450 nm). The plate was read at 450 nm using an absorbance plate reader.

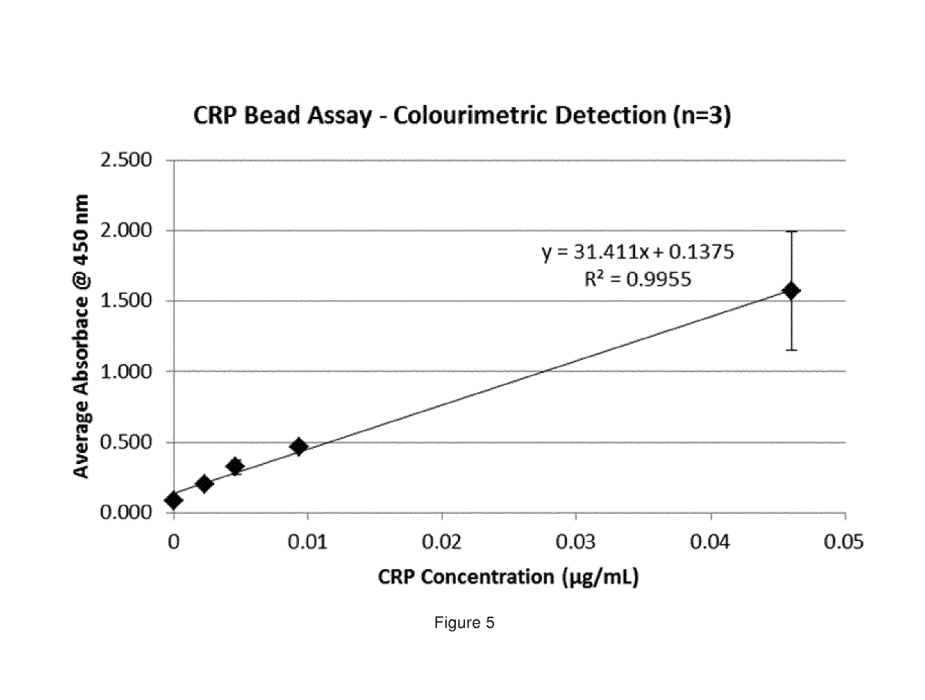

The results (FIG. 5) demonstrated that the assay was functioning correctly and a linear trend for increasing concentration of CRP was observed.

5. Electrochemical Detection of CRP Bead Assay with Washing

The CRP bead assay was tested with electrochemical detection. The method for the colourimetric assay was followed, only substituting the colourimetric detection method with an electrochemical detection method.

The analyte was incubated for 1 hour with the detection antibody which was labelled with HRP in a reaction chamber. Magnetic beads that were conjugated with capture antibody were then added to the reaction chamber and the solution was incubated for an additional hour. The magnetic beads were washed three times. The test solution was then diluted with electrochemical buffer and pipetted onto a well on the electrode sensor and a measurement was made at 30 mV vs. Ag/AgCl REF for 30 seconds.

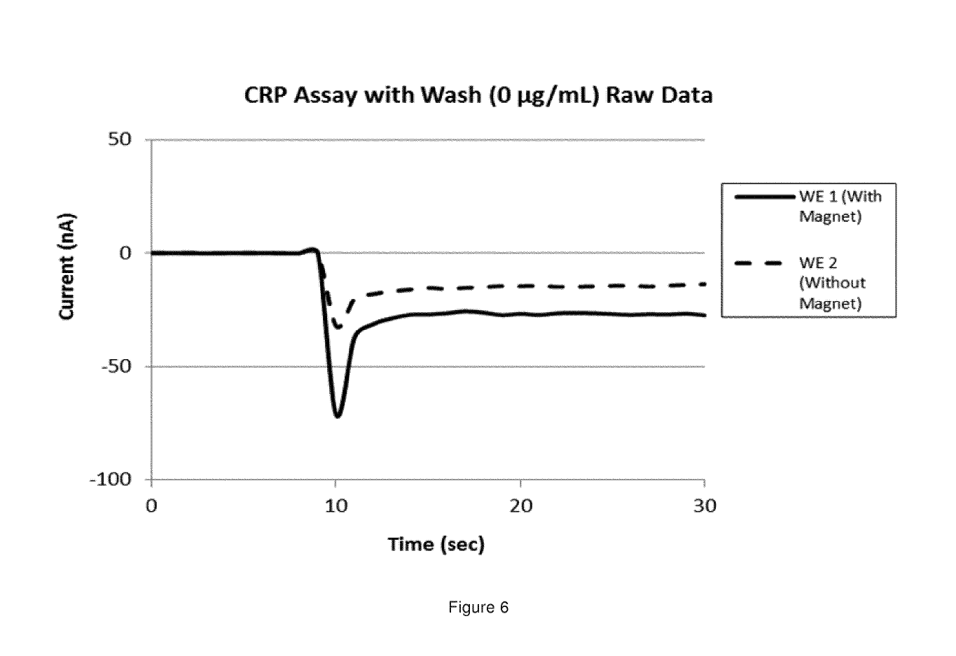

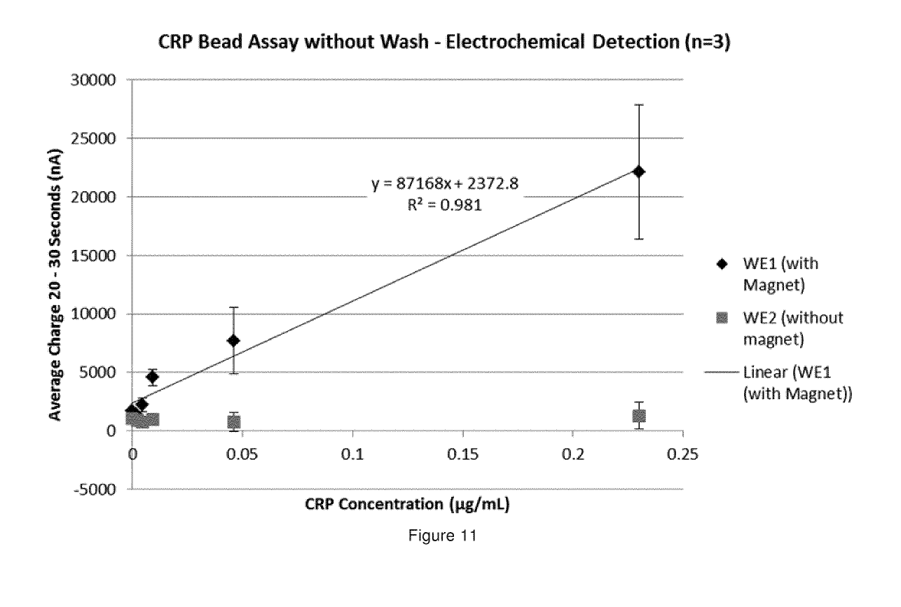

Current versus time graphs of examples of the raw data for 0 μg/mL and 0.23 μg/mL CRP for an assay where the beads were washed prior to electrochemical detection are shown in FIGS. 6 & 7 respectively. The full results of the assay which are given as charge vs. CRP concentration are shown in FIG. 11. The results demonstrate that the bead assay and electrochemical detection can be successfully combined.

6. Electrochemical Detection of CRP Bead Assay without Washing

Experiments were then performed without a wash step. The analyte was incubated for 1 hour with the HRP labelled detection antibody in a reaction chamber. Magnetic beads conjugated with capture antibody were then added to the reaction chamber and the solution was incubated for an additional hour. The solution was removed from the reaction chamber and diluted with the electrochemical buffer. The test solution was pipetted onto a well on the electrode sensor and a measurement was made at 30 mV vs. Ag/AgCl REF for 30 seconds.

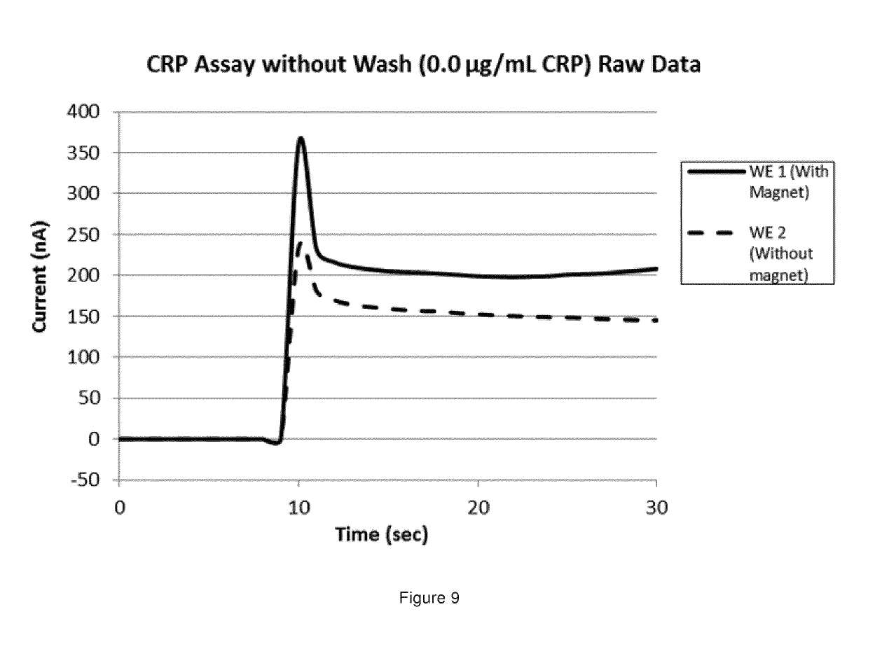

Current versus time graphs of examples of the raw data for 0 μg/mL and 0.23 μg/mL CRP are shown in FIGS. 9 & 10 respectively. The full results of the assay which are given as charge vs. CRP concentration are shown in FIG. 11. The charge was calculated using the rectangular rule for the measurement period from 20-30 seconds. The results demonstrated that the bead assay and electrochemical detection could be successfully combined without the requirement for a wash.

1. A method for detecting analyte in a sample comprising:
a) providing an electrochemical sensor comprising;
a control sensing element,
a detection sensing element and
a magnet which selectively attracts magnetic beads to the detection sensing element relative to the control sensing element,
wherein upon exposure to a detection solution, each sensing element produces a signal which is indicative of the amount of an enzymatic label present in the detection solution at the sensing element,
b) exposing the sensing elements to a detection solution,
wherein the detection solution comprises;
i) a sample being tested for the presence of analyte
ii) a detection antibody reactive with said analyte which is attached to the enzymatic label, and
iii) a separation antibody reactive with said analyte which is attached to a magnetic bead,
such that, in the presence of analyte in the sample, a immunocomplex is formed in the detection solution which comprises the analyte, the detection and separation antibodies, the enzymatic label and the magnetic bead, and;
c) measuring the signals produced by the detection and control sensing elements, and
d) determining from said signals the amount of enzymatic label in the detection solution at the detection sensing element and the control sensing element,
wherein an increase in the amount of enzymatic label at the detection sensing element relative to the control sensing element is indicative of the presence or amount of analyte in the sample.

2. A method according to claim 1 wherein the magnet attracts magnetic beads to the detection sensing element and does not attract magnetic beads to the control sensing element.

3. A method according to claim 1 or claim 2 wherein the magnet is located at the detection sensing element.

4. A method according to any one of claims 1 to 3 wherein
each said sensing element comprises a working electrode, a counter electrode, and optionally a reference electrode,
each working electrode having an electrically conductive matrix holding a first reagent and/or a second reagent, the second reagent being an oxidising agent or a precursor thereof for the first reagent;
wherein the electrically conductive matrix is an electrically conductive carbon- or graphite-containing matrix or an electrically conductive porous matrix and a reaction between the first reagent and the oxidising agent is catalysable by an enzymatic label to provide a detectable signal at the working electrode.

5. A method according to claim 4 wherein the signals produced by the detection and control sensing elements are measured by maintaining a potential between the working electrodes and the counter electrode and/or the reference electrode, where present, in the detection and control sensing elements; and measuring the currents passing between the test and control working electrodes and the counter and/or reference electrode where present, in the detection and control sensing elements.

6. A method according to claim 4 or claim 5 wherein an increase in the amount of current passing between the working electrode and the counter, and/or reference electrode where present, in the detection sensing elements relative to the amount of current passing between the control working electrode and the counter and/or reference electrode where present, in the control sensing element is indicative of the presence or amount of analyte in the sample.

7. A method according to any one of claims 4 to 6 wherein the electrically conductive matrix is a carbon paste.

8. A method according to any one of claims 4 to 7 wherein the first reagent is tetramethylbenzidine.

9. A method according to any one of claims 4 to 8 wherein the second reagent is perborate.

10. A method according to any one of the preceding claims wherein the sensor comprises a detection chamber which accommodates the detection solution.

11. A method according to any one of the preceding claims wherein the detection antibody is covalently linked to the enzymatic label.

12. A method according to any one of claims 1 to 10 wherein the detection antibody is non-covalently linked to the enzymatic label.

13. A method according to claim 12 wherein the detection antibody is linked to the e enzymatic label though a secondary antibody

14. A method according to any one of the preceding claims wherein the separation antibody is covalently linked to the magnetic bead.

15. A method according to any one of claims 1 to 14 wherein the separation antibody is non-covalently linked to the magnetic bead.

16. A method according to claim 15 wherein the separation antibody is linked to the magnetic bead through a secondary antibody

17. A method according to any one of the preceding claims wherein the enzymatic label is peroxidase, for example horseradish peroxidase (HRP).

18. A method according to any one of the preceding claims wherein t the detection solution is produced by admixing the sample, the detection antibody attached to the enzymatic label, and the separation antibody attached to a magnetic bead in an assay solution and modifying the assay solution to produce the detection solution.

19. A method according to claim 18 wherein sample the detection antibody attached to the enzymatic label, and the separation antibody attached to the magnetic bead are incubated for 2-5 minutes in the assay solution at pH 7 to 8.

20. A method according to claim 18 or claim 19 wherein the assay solution is modified by reducing the pH to produce the detection solution.

21. An electrochemical sensor for detecting analyte in a solution comprising;
a control sensing element,
a detection sensing element and
a magnet which selectively attracts magnetic beads in the solution to the detection sensing element relative to the control sensing element,
such that upon exposure to a solution, each said sensing element produces a signal which is indicative of the amount of enzymatic label present in the solution at the sensing element.

22. A sensor according to claim 21 wherein the magnet attracts magnetic beads to the detection sensing element and does not attract magnetic beads to the control sensing element.

23. A sensor according to claim 21 or claim 22 wherein the magnet is located at the detection sensing element.

24. A sensor according to any one of claims 21 to 23 wherein
each said sensing element comprises a working electrode, a counter electrode, and optionally a reference electrode,
each working electrode having an electrically conductive matrix holding a first reagent and/or a second reagent, the second reagent being an oxidising agent or a precursor thereof for the first reagent;
wherein the electrically conductive matrix is an electrically conductive carbon- or graphite-containing matrix or an electrically conductive porous matrix and a reaction between the first reagent and the oxidising agent is catalysable by an enzymatic label to provide a detectable signal at the working electrode.

25. A sensing device comprising;
an electrochemical sensor according to any one of claims 1 to 24,
a sampler for accommodating a sample from an individual and introducing said sample to the electrochemical sensor,
an electronic reader for determining from signals produced by the sensing elements of the electrochemical sensor the amount of analyte in the sample and providing an output indicative of said amount.

26. A kit for detecting an analyte comprising;
an electrochemical sensor according to any one of claims 21 to 24 or a sensing device according to claim 25,
a detection antibody reactive with said analyte which is attached or attachable to an enzymatic label;
a separation antibody reactive with said analyte which is attached or attachable to a magnetic bead, and; optionally one or more buffers or other reagents.

27. Use of an electrochemical sensor according to any one of claims 21 to 24, a sensing device according to claim 25 or a kit according to claim 26 in a method for detecting analyte.

28. Use according to claim 27 wherein the method is a method according to any one of claims 1 to 20.
